(19) United States
(12) Patent Application Publication
(10) Pub. No.: US 2021/0019581 A1
(43) Pub. Date: Jan. 21, 2021

(54) LABELLING SYSTEM AND METHOD

(71) Applicant: CLINICAL SYSTEMS, INC, Garden City, NY (US)

(72) Inventors: Roger Halvorsen, Garden City, NY (US); Chandresh Mody, Monmouth, NJ (US)

(73) Assignee: CLINICAL SYSTEMS, INC, Garden City, NY (US)

(21) Appl. No.: 16/932,308

(22) Filed: Jul. 17, 2020

Related U.S. Application Data
(60) Provisional application No. 62/875,688, filed on Jul. 18, 2019.

(51) Int. Cl. G06K 15/02; G06F 40/55; G16H 10/20

(52) U.S. Cl. CPC G06K 15/024; G16H 10/20; G06F 40/55

(57) ABSTRACT

Methods and systems related to clinical studies are disclosed. For example, the system may comprise a memory storing study data for a particular clinical study. The study data may be stored in a plurality of languages. The system may also comprise an encoder which may be configured to receive an identifier associated with the particular clinical study and one or more identifiers identifying portions of the study data and encode a tag with the identifier associated with the particular clinical study and identifiers identifying portions of the study data, a printer which may be configured to print text and/or graphics on a label and a processor. The label has the tag. In response to the tag being read by a terminal, the processor is configured to transmit the portions of the study data identified by the one or more identifiers in a language associated with the portions after authentication.

### CROSS REFERENCE TO RELATED APPLICATIONS

This application claims the benefit of Provisional Application No. 62/875,688, filed on Jul. 18, 2019, the entire contents of which are incorporated by reference.

### BACKGROUND

Typical booklet labels for use with clinical trials must include several pages of information. Especially in double blind trials, the booklet label provided on the medication package must provide detailed instructions and information on how the medication is to be stored, delivered, and other concerns, such as time of day the medication is to be delivered and whether any food and/or drinks are to be avoided or consumed in a specified time frame around the delivery. Additionally, typical booklet labels must include information about the lot the medication within the package is part of. Also, many typical booklet labels include a “blinded” portion, which is initially covered by a substance to avoid view by the clinician and/or subject, and can later be removed by a mechanical and/or chemical process for viewing.

As a further complication of typical booklet labels, usually clinical trials are conducted in many jurisdictions with many languages. Thus, proper translations, printing and shipping of specific labels for specific jurisdictions must be coordinated and performed.

Therefore, what is desired is a system and method of printing a label for each trial language, which only contains limited typographical information, but includes a way for a clinician with a device to access a database that includes all relevant information about the package the label is attached to.

### SUMMARY OF THE DISCLOSURE

Accordingly disclosed is a system which may comprise a memory, an encoder, a processor, and a printer. The memory may be configured to store study data for a particular clinical study. At least a portion of the study data may be stored in a plurality of languages. The encoder may be configured to receive an identifier associated with the particular clinical study and one or more identifiers identifying portions of the study data and encode a tag with the identifier associated with the particular clinical study and one or more identifiers identifying one or more portions of the study data.

The printer may be configured to print text and/or graphics on a label. The label may have the tag. In response to the tag being read by a terminal, the processor may be configured to transmit the one or more portions of the study data identified by the one or more identifiers in a language associated with the one or more portions after authentication.

In an aspect of the disclosure, the processor may transmit a list of the plurality of languages and the language transmitted to the terminal by the processor may be based on a user selection on the list.

In an aspect of the disclosure, the language transmitted by the processor may be based on a geographic location of the terminal.

In other aspects of the disclosure, the identifiers identifying portions of the study may further indicate the language. In other aspects of the disclosure, the tag may comprise a tag identifier and the tag identifier identifies the language.

In an aspect of the disclosure, the identifier associated with the particular clinical study may comprise a first portion which causes the terminal to authenticate. The first portion may be a universal resource locator (URL) associated with the particular clinical study.

In an aspect of the disclosure, the encoder may be configured to encode a plurality of tags for the particular clinical study. The plurality of tags may comprise a first group of tags and a second group of tags. The language transmitted for the first group of tags may be different from the language transmitted for the second group of tags.

In aspect of the disclosure, the encoder may be further configured to encode additional portions of the study data in the tag. The additional portions of the study data may be encoded in one or more languages associated with the portions.

In an aspect of the disclosure, the study data may comprise one or more of a clinical study name, a container type a generated label will be attached to, a size and a shape of the generated label, design elements of the generated label, logic to encode the tag, regimen tables, lot sets, manufacturing batch data, manufacturing packaging lots, randomization data, storage instructions, lot information, predefined phrases and terminology, and sequential numbering of individual labels.

Also disclosed is a method which may comprise storing study data in a memory for a particular clinical study, at least a portion of the study data being stored in a plurality of languages, encoding a tag with an identifier associated with the particular clinical study and one or more identifiers identifying one or more portions of the study data to form an encoded tag, printing a label having the encoded tag; and in response to a terminal reading the encoded tag, transmitting the one or more portions of the study data identified by the one or more identifiers in a language associated with the one or more portions after authentication.

In an aspect of the disclosure, the encoded tag may cause the terminal to be directed to a location to authenticate.

In an aspect of the disclosure, the method may further comprise in response to the terminal reading the encoded tag, transmitting a list of the plurality of languages to the terminal for selection and in response to receiving the selection, transmitting the portions of the study data identified by the one or more identifiers in the selected language.

In an aspect of the disclosure, the method may further comprise receiving a list of target languages from a pharmaceutical company managing the particular clinical study and providing translations of the study data in the target languages as the plurality of languages.

In an aspect of the disclosure, the method may further comprise generating the study data by creating a model.

In an aspect of the disclosure, the method may further comprise determining a location of the terminal and transmitting the portions of the study data identified by the one or more identifiers in a language based on the determined location.

In other aspects of the disclosure, the identifiers identifying the one or more portions of the study may further indicate the language. In other aspects of the disclosure, the tag may comprise a tag identifier and the tag identifier identifies the language.

In an aspect of the disclosure, the method may further comprise encoding portions the study data into the tag.

In an aspect of the disclosure, the study data may be encoded in one or more languages associated with the one or more portions.

In an aspect of the disclosure, the method may further comprise encoding a plurality of tags for the particular clinical study. The plurality of tags may comprise a first group of tags and a second group of tags. The language transmitted for the first group of tags may be different from the language transmitted for the second group of tags.

### BRIEF DESCRIPTION OF THE DRAWINGS

These and other features, aspects, and advantages of the present disclosure will become better understood with regard to the following description, appended claims, and accompanying drawings wherein:

FIG. 1 is a system diagram of an embodiment of a system.

FIG. 2 is a flow chart illustrating a portion of the method.

FIG. 3 is a flow chart illustrating a portion of the method.

FIG. 4 is a flow chart illustrating a portion of the method.

### DETAILED DESCRIPTION

As used herein, the term “RFID tag” will refer to any active or passive type of electronic data storage device, read-only or read and write, that is wirelessly activated in the presence of a radio frequency (RF) field, including any inductively coupled RFID tags, capacitively coupled RFID tags and even future RF-type tags not yet available.

As used herein, the term “tag” or “tags” refer to “RFID tags” as well as other data storage devices that can be accessed through other electronic means, other than radio frequencies. Some examples of these “tags” include but are not limited to ultra wide-band (UWB) real-time location systems (RTLS), WiFi RTLS and infrared RTLS.

Also, the tag may be a silicon-type IC tag, a printed tag printed with a conductive ink-based printing process or a tag formed by other suitable means.

RFID tags are electronic circuits that typically consist of a coil that acts as an antenna and a small silicon-based microprocessor with a memory, all optionally encapsulated in a sealing material. RFID tags store identification information, usually in the form of an identification number that corresponds to an object or item to which the tag is attached.

When a tag enters an RF field generated by a reader, the circuit of the tag becomes energized causing the processor to perform a data operation, usually by emitting a signal containing the processor's stored information. As used herein, the term “reader” or “tag reader” refers to any device that can emit an RF field (such as a Bluetooth® enabled device and/or a Near Field Communication (NFC) device). The reader can be stationary, or the reader can be a mobile device, such as a smart phone, tablet or other portable device that either contains a power source or can receive a power input, which can emit an RF field.

Tags can be formed on a substrate, such as, a paper and/or fabric containing material, and can include analog RF circuits, digital logic, and memory circuits. Tags also can include a number of discrete components, such as capacitors, transistors, and diodes. Tags can be categorized as either active or passive.

Active tags have their own discrete power source such as a battery. When an active tag enters an RF field it is turned on and then emits a signal containing its stored information.

Passive tags do not contain a discrete power source. Rather, they become inductively or capacitively charged when they enter an RF field. Once the RF field has activated the passive circuit, the passive tag emits a signal containing its stored information.

In addition to retrieving and transmitting data previously stored in the memory, both passive and active dynamic RFID tags and tags can permit new or additional information to be stored in the RFID tag's memory or tag's memory, or can permit the RFID tag, or tag, to manipulate data or perform some additional functions. Alternatively, in addition to retrieving and transmitting data previously stored in the memory, both passive and active dynamic RFID tags and tags can block new or additional information to be stored in the RFID tag's, or tag's, memory, and/or can block the RFID tag or tag from having any data contained therein from being manipulated.

Tags, both active and passive, generally come in one of two configurations: inductively or capacitively coupled. Inductively coupled tags consist of a silicon-based microprocessor, a metal coil wound into a circular pattern that serves as the tag's antenna, and an encapsulating material that wraps around the chip and coil. These tags are powered by an electromagnetic field generated by the tag reader, which is received by the tag's antenna.

The second type of tags are capacitively coupled tags. Capacitively coupled tags do not include the metal coil, consisting instead of a silicon microprocessor, paper substrate, and a conductive carbon ink that is applied to the paper substrate through printing.

The tags discussed above can be incorporated into or on at least a portion of a label.

As used herein, the term label “label” is intended to include a label, strip, tag or general display device that is sufficiently sized and configured for attachment to a medication maintaining container (such as but not limited to a bottle, a vial, a box, a foil and plastic medication holder, or any other suitable container that can maintain one or more of a solid, liquid and gas medication).

As used herein, “medication” refers to any solid, liquid or gas material that does contain a medicament or does not contain a medicament (such as in a placebo). The medicament includes any drug, vaccine, or any other material that is capable of causing a reaction in a subject's body (for example stem cells, radiation, magnetism, etc.)

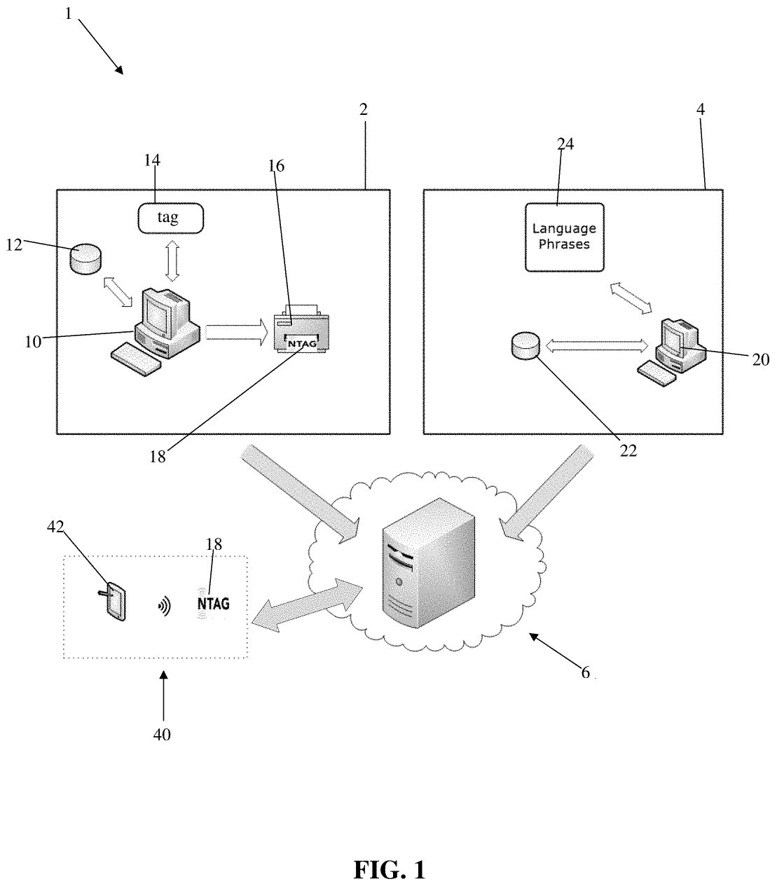

Referring now to FIG. 1, a system 1 in accordance with aspects of the disclosure is shown. The system 1 includes several components, including a label generating system 2, a translation system 4, and a server 6. The server 6 comprises a memory and a processor.

In the label generating system 2 a computer 10 (which includes a processor and other computing components, such as a memory, etc., and can include any input device such as a mouse and/or keyboard) communicates with both a database 12 and a tag 14. Although database 12 is shown as being separate from the computer 10, database 10 can be a component of the computer 10. The database 12 can store labeling models and study data, as discussed below. All arrows of communication shown in the label generating system 2 (as well as in the rest of the application) represent either any suitable wired or any suitable wireless communication between components.

In label generating system 2, a user interacts with the computer 10 to create a model of for the clinical study in order to generate study data. In an aspect of the disclosure, the model may be created based on information provided by a pharmaceutical company or agent managing, directing or designing the clinical study. This study data can include, amongst other information, one or more of a identifiers for the clinical study, e.g., a name, container type the generated label will be attached to, size and shape of the generated label, design elements (including placement of text and/or images such as logs) of the generated label, logic to encode the tag 14, regimen tables that include the sequence and frequency of labeling and packaging hierarchy, lot sets that define the medicament product, manufacturing batch data and packaging lots, randomization data that defines the Material and Patient randomization information, generation specifications that control the sequence that labels are generated by the system, variable data associations from all the pertinent database elements in the design, usage instructions, storage instructions, lot information, sequential numbering of individual labels (including individual unit identifying values for the individual label), etc. for a number of labels, which will subsequently be printed. The study data may also include the phrases and terminology associated with the identifiers (placeholders described herein). This study data can also include various levels of accessibility of information based on the level of clinician user. For example, a clinician user that is a doctor may be provided many or all details regarding the medication, while a clinician user that is a nurse may be provided less details, such as only details regarding storage and instructions for administration.

Once a study data is created the computer 10 stores the data associated with the study data and/or transmits the data associated with the study to the server 6, and the computer 10 communicates with an encoder to encode the tag 14 with data that can allow for access to the data on the server 6 by a clinician user.

The computer 10 is also configured to communicate with a printer 16, which itself is configured to print text and/or graphics on a label 18 the tag 14 is attached to. To perform the printing process on the label 18, the printer 16 is sent generation specifications from the computer 10. These generation specifications have been created by a user of the computer 10 after the study data is stored in server 6. The generation specifications control the output of all study data, to be output from the printer 16 onto the label 18, as well as the encoding of the tag 14. These generation specifications include what text is to be printed on to the label 18 by the printer 16, in the specific language desired.

In some embodiments the tag 14 is then attached to the label 18 after the printer 16 prints the text and/or graphics on the label 18. In other embodiments, the tag 14 is already attached to label 18, prior to printing, and the tag 14 is encoded while it is attached to the label 18 either before or after the printer 16 prints the text and/or graphics.

The translation system 4 includes a computer 20 (which may be the same as the computer 10, or a different device) which can access and retrieve information from a language database 22 and a language phrase repository 24.

Based on the specific requirements of the study and any company specific requirements, a number of placeholders (also referenced as “identifiers”) are created for phrases and terminology (also referenced as “portions of the study data”) that is stored in server 6, discussed below. For example, the phrase “For Clinical Trial Use Only” may be indicated by a unique identifier “16”. The identifier may represent the number of the phrase. In other aspects, the identifier may be “#16 English”, which indicates both the number of the phrase and language. In other aspects of the disclosure, a different identifier may be used such as a location identifier indicated a stored location within the server 6 for the phrase, e.g., the portion of the study data. Thus, the phrase “For Clinical Trial Use Only” may be viewable by the clinician user in their own language, based on country the label is to be sent to and/or the language that the clinician user speaks, as discussed below. The user can create many identifiers, for each desired language and phrase, e.g., portion of the study data, to be sent to the server 6, which can be referred to as discussed below. For example, in an aspect of the disclosure, for the same phrase, e.g., portion of the study data, different identifiers may be created for the different available languages. In an aspect of the disclosure, the pharmaceutical company or agent managing, directing or designing the clinical study, may determine the number and specific languages available for the clinical study and input the same into the system 1.

In an aspect of the disclosure, the system 1 contains a language phrase repository 24. The language phrase repository 24 contains available phrases and terminology for any clinical study in the available language(s), which is selectable. However, for a particular study only a subset of the portions (phrases and terminology) and a subset of the language may be used or selected for use. The language selected for use may be based on the expected countries where subjects in the clinical study are located. In an aspect of the disclosure, the selected phrases and terminology, e.g., portions, may also be determined by the above described model in combination with information provided by the pharmaceutical company or agent managing, directing or designing the clinical study. For example, if the countries to include are France and the United States, the available languages may include French and English, among other languages. Therefore, the study data may be stored in the different languages.

The language phrase repository 24 can then create a global data set, for all languages selected, and for the phrases associated with the identifiers for the particular clinical study, and store that global data set in the language database 22. This global dataset stored in the language database 22 would contain similar information as to the universe of information now provided in text form on a booklet label. In some aspects of the disclosure, the global data set is created when the model is created. In other aspects of the disclosure, the global data set is created when the tags are encoded.

At a time, the global data set in the language database 22 is transferred to the server 6. Prior to a time when the labels would be accessed by users in a clinical setting.

Once the label 18 and associated medical container arrive at a clinical site 40, the clinician user may operate a reader 42 to access the data on the tag 16 of the label 18.

Once the data on the tag 16 is accessed by the reader 42, the reader 42 interacts with the server 6 and the reader 42 is directed to a specific interface (or website, or application based interface), which can be hosted by the server 6, or be a remotely hosted interface. In an aspect of the disclosure, the tag may cause the terminal associated with the reader to be redirected for authentication. Authentication may be performed using a website hosted by the pharmaceutical company managing the clinical study or another agent. In an aspect of the disclosure, the tag 16 may include an URL for the website for authentication. In an aspect of the disclosure, the URL may include the identifier of the clinical study. In other aspects, the identifier of the clinical study may be separately encoded. In an aspect of the disclosure, an identifier associated with the pharmaceutical company may also be used. The specific interface will visually display (or be provided as a file for print or use by a suitable device) the clinician accessible set of data associated with the identifier(s) on the tag 16 in a language as described below.

The server 6 can provide the clinician user the clinician accessible set of data in the clinician user's own language in one or more of several ways.

A first way the appropriate language can be determined is the appropriate language can be affiliated with a range of identification numbers that can be printed on the label 18 and/or be included in the data on the tag 16. For example, for the range of identification numbers 4,001 to 5,000, the affiliated language will be French, for the range of identification numbers 5,001 to 6,000 the affiliated language will be English.

A second way the appropriate language can be determined is through a prompt received by the clinician user through the reader 42. For example, once the reader 42 accesses the data on the tag 16, the clinician user is provided with a list of languages to choose from. Once the language is chosen, and the chosen language is received by the server 6, the server 6 can then provide the clinician accessible set of data in the selected language.

A third way the appropriate language can be determined is through a location based determination of the reader 42. If the reader 42 includes a location feature, which can locate the placement of the reader 42 geographically on Earth, the reader 42 can transmit the location of the reader 42 to the server 6. Once the location is received, the server 6 can either (1) automatically select a language to transmit the clinician accessible set of data in (for example, if the location of the reader 42 is in Japan, the automatic language transmitted is Japanese) or (2) provide an abbreviated list of languages for the clinician user to select from (for example, if the location of the reader 42 is Canada, the abbreviated list of languages can include English and French).

In other aspects, the tag may include a location identifier pointing to the portion of the study data and specific language in the server 6 (e.g., the phrase and terminology associated with the identifier).

In other aspects of the disclosure, the tag 16 may include other study data, e.g., other portions of the study data. In an aspect of the disclosure, the other study data may also be provided in an appropriate language.

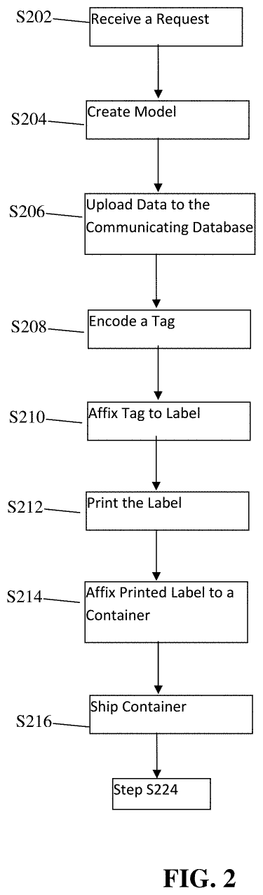

A method of forming and using these labels is discussed below in reference to FIG. 2.

Initially, in step S202, a request is received from an entity, a pharmaceutical company for example, to conduct a particular clinical study, with specific parameters, for a specific medicament in specific countries.

Next, in Step S204, a user accesses the computer 10 and inputs the requested information received in S202, to create a model for the particular clinical study which generates the study data.

Next, in S206, the study data is uploaded to the communicating server 6, for storage in the server 6 and future access by a clinician.

Then, in step S208, the tag 14 is encoded with, at the least, an identifier of the clinical study such as a name, and identifiers of the portions (phrases and terminology) of the study data. Then, the tag 14 is affixed to a label 18 in step S210. The tag 14 can be affixed to the label 18 in any suitable way, such as through an adhesive.

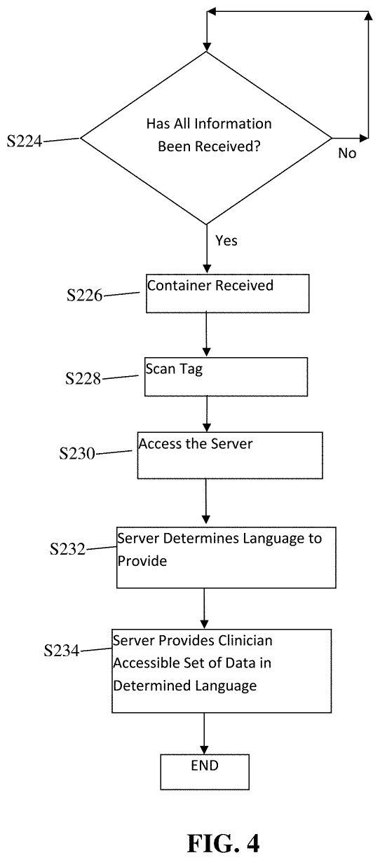

Once the tag 14 is affixed to the label 18, the label 18 has text and/or images printed onto the label 18 in S212. After printing, the label 18 that is printed upon (and the affixed tag 14) are affixed to a container in S214. The label 18 can be affixed to the container in any suitable way, such as through an adhesive, or placement of the label 18 into a formed opening or pocket of the container. The container having the affixed label 18 and tag 14 can then be shipped in any suitable way to a site the clinical test is to occur in Step S216. After shipment, the method proceeds to Step S224 in FIG. 4.

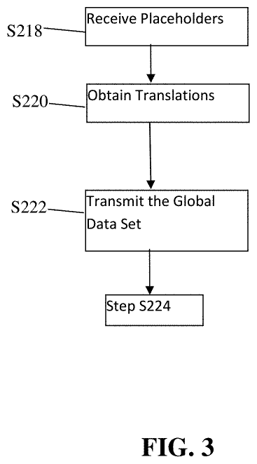

Separately, another set of steps is illustrated in FIG. 3. The steps in FIG. 3 can occur at any time after S202 of FIG. 2.

Next, phrases and terminology stored in the translation system 4 may be selected (and become portions of the study data), and an identifier(s) associated with the selected phrases and terminology (portions of the study data) in step S218. In an aspect of the disclosure, the user may access the translation system 4 to select the phrases and terminology from the language phrase repository (and select the language). In other aspects of the disclosure, the system 1 may automatically select the phrases and terminology (and languages) from the language phrase repository 24 based on the model. Then, the language phrase repository 24 is accessed by the computer 20 for receiving a translation in selected languages for each placeholder in S220, creating a global data set, of each placeholder in all selected languages, that is saved in the language database 22.

Next, the global data set is transmitted to the server 6 in S222. After transmission, the method proceeds to Step S224 in FIG. 4.

At any time after Steps S216 and S222 the server 6 can check to ensure that both information received from the label generating system 2 and the information received from the translation system 4 have been received in Step S224. If not, the method can loop back and the communicating server 6 can continue to check if the information has been received. If yes, the method can proceed to Step S226, receipt of the container at the clinical site 40.

After receipt of the container, with the label 16 and tag 14 affixed thereto, after a period of time, a clinician is ready to begin the trial at the clinical site 40. To begin, the clinician scans the tag 14 with their reader 42 in step S228.

Next, in step S230, the reader 42, based on the information received from scanning of the tag 14, accesses the server 6 in Step S230.

The server 6 then determines what language to provide a portion of the global data set to the reader 42 based on a received input in Step S232. This received input can be a selection by the clinician user made on the reader 42 and/or a determination of the location of the reader 42 and/or a determination of the language specified by an indicator of language in the data of tag 14.

Next, in Step 234, the server 6 provides the portion of the global data set as a clinician accessible set of data, in the language determined in Step 232, to the terminal associated with the reader 42 (to appear on a display of the reader 42 and/or be printed at the clinical site 40).

Then, after Step S234, the clinician can review the provided clinician accessible set of data and dispense the medicament from the container and/or properly store the container for later use.

Many of the above described steps may be reversed in order to arrive at the same endpoint. For example, S208 and S210 can be reversed before proceeding with the same S212.

The computers 10 and 20, the server 6, and databases 12, and 22, and reader 42 may be any type of known or will be known systems and may include one or more of processors, memory devices, storage devices, input/output devices, internal buses, and/or communications interfaces for communicating with other computer systems in conjunction with communication hardware and software, etc. Portions of the systems also may be implemented on a virtual computer system, colloquially known as a cloud.

Regarding a computer readable storage medium, it may be, for example, a magnetic, optical, electronic, electromagnetic, infrared, or semiconductor system, apparatus, or device, or any suitable combination of the foregoing; however, the computer readable storage medium is not limited to these examples. Additional particular examples of the computer readable storage medium can include: a portable computer diskette, a hard disk, a magnetic storage device, a portable compact disc read-only memory (CD-ROM), a random access memory (RAM), a read-only memory (ROM), an erasable programmable read-only memory (EPROM or Flash memory), an electrical connection having one or more wires, an optical fiber, an optical storage device, or any appropriate combination of the foregoing; however, the computer readable storage medium is also not limited to these examples. Any tangible medium that can contain, or store a program for use by or in connection with an instruction execution system, apparatus, or device could be a computer readable storage medium.

The described embodiments of the present disclosure are intended to be illustrative rather than restrictive, and are not intended to represent every embodiment of the present disclosure. Various modifications and variations can be made without departing from the spirit or scope of the disclosure as set forth in the following claims both literally and in equivalents recognized in law.

What is claimed is:

1. A system comprising:
a memory configured to store a study data for a particular clinical study, at least a portion of the study data being stored in a plurality of languages;
an encoder configured to receive an identifier associated with the particular clinical study and one or more identifiers identifying one or more portions of the study data and encode a tag with the identifier associated with the particular clinical study and the one or more identifiers identifying the one or more portions of the study data;
a processor; and
a printer configured to print text and/or graphics on a label, the label having the tag,
wherein in response to the tag being read by a terminal, the processor is configured to transmit the one or more portions of the study data identified by the one or more identifiers in a language associated with the one or more portions after authentication.

2. The label system of claim 1, wherein the processor transmits a list of the plurality of languages and the language transmitted by the processor is based on a user selection on the list.

3. The label system of claim 1, wherein the language transmitted by the processor is based on a geographic location of the terminal.

4. The label system of claim 1, wherein the identifiers identifying the one or more portions of the study further indicates the language.

5. The label system of claim 1, wherein the tag comprises a tag identifier and the tag identifier identifies the language.

6. The label system of claim 1, wherein the identifier associated with the particular clinical study comprises a first portion which causes the terminal to authenticate.

7. The label system of claim 6, wherein the first portion is a universal resource locator associated with the particular clinical study.

8. The label system of claim 1, wherein the encoder is configured to encode a plurality of tags for the particular clinical study, the plurality of tags comprising a first group of tags and a second group of tags, wherein the language transmitted for the first group of tags is different from the language transmitted for the second group of tags.

9. The label system of claim 1, wherein encoder is further configured to encode portions of the study data in the tag.

10. The label system of claim 9, wherein the study data is encoded in one or more languages associated with the portions.

11. The system of claim 1, wherein the study data comprises one or more of a clinical study name, a container type a generated label will be attached to, a size and a shape of the generated label, design elements of the generated label, logic to encode the tag, regimen tables, lot sets, manufacturing batch data, manufacturing packaging lots, randomization data, storage instructions, lot information, predefined phrases and terminology, and sequential numbering of individual labels.

12. A method comprising:
storing study data in a memory for a particular clinical study, at least a portion of the study data being stored in a plurality of languages;
encoding a tag with an identifier associated with the particular clinical study and one or more identifiers identifying one or more portions of the study data to form an encoded tag;
printing a label having the encoded tag; and
in response to a terminal reading the encoded tag, transmitting the one or more portions of the study data identified by the one or more identifiers in a language associated with the one or more portions after authentication.

13. The method of claim 12, wherein the encoded tag causes the terminal to be directed to a location to authenticate.

14. The method of claim 12, further comprising in response to the terminal reading the encoded tag, transmitting a list of the plurality of languages to the terminal for selection and in response to receiving the selection, transmitting the portions of the study data identified by the one or more identifiers in the selected language.

15. The method of claim 12, further comprising receiving a list of target languages from a pharmaceutical company managing the particular clinical study and providing translations of the study data in the target languages as the plurality of languages.

16. The method of claim 12, further comprising generating the study data by creating a model.

17. The method of claim 12, further comprising determining a location of the terminal and transmitting the portions of the study data identified by the one or more identifiers in a language based on the determined location.

18. The method of claim 12, wherein the identifiers identifying the one or more portions of the study further indicates the language.

19. The method of claim 12, wherein the tag comprises a tag identifier and the tag identifier identifies the language.

20. The method of claim 12, further comprising encoding portions the study data into the tag.

21. The method of claim 20, wherein the study data is encoded in one or more languages associated with the portions.

22. The method of claim 12, further comprising encoding a plurality of tags for the particular clinical study, the plurality of tags comprising a first group of tags and a second group of tags, wherein the language transmitted for the first group of tags is different from the language transmitted for the second group of tags.
